(19) United States
(12) Patent Application Publication
(10) Pub. No.: US 2009/0304698 A1
(43) Pub. Date: Dec. 10, 2009

(54) MODULATION OF OVULATION

(75) Inventors: Jennifer Lee Juengel, Upper Hutt (NZ); Kenneth Pattrick McNatty, Upper Hutt (NZ); Lloyd Gary Moore, Upper Hutt (NZ); Robert Syndecombe Bower, Dunedin (NZ)

Correspondence Address: KNOBBE MARTENS OLSON & BEAR LLP, 2040 MAIN STREET, FOURTEENTH FLOOR, IRVINE, CA, 92614, US

(73) Assignee: AGRESEARCH LIMITED, Hamilton (NZ)

(21) Appl. No.: 11/720,827

(22) PCT Filed: Dec. 2, 2005
(86) PCT No.: PCT/NZ05/00313

§ 371 Date: Oct. 26, 2007

(30) Foreign Application Priority Data
Dec. 2, 2004 (NZ) 536943
Mar. 24, 2005 (NZ) 539-39
Jul. 8, 2005 (NZ) 541170

(51) Int. Cl. A61K 39/395; C07K 14/47; C07K 7/08; C07K 16/18; A61K 38/17; A61K 38/10

(52) U.S. Cl. 424/139.1; 530/324; 530/327; 530/387.9; 514/12; 514/14

(57) ABSTRACT

The present invention is directed to the identification of a novel domain of functional significance on the GDF-9 and GDF-9B molecules and to peptides and antibodies which interact with the domain to modulate the biological activity of these molecules to alter mammalian ovarian function and ovulation rate.

### FIELD OF THE INVENTION

The present invention relates the identification of a novel domain of functional significance on GDF-9 and GDF-9B molecules and to agonists and antagonists which interact therewith to modulate the biological activity of these molecules to alter mammalian ovarian function and ovulation rate.

### BACKGROUND OF THE INVENTION

GDF-9 and GDF-9B (also known as BMP15) are expressed in the oocyte of the developing follicle and play a role in mammalian fertility (Fitzpatrick et al, 1998). GDF9 is a member of the transforming growth factor beta (TGFβ) superfamily (McPherron and Lee, 1993) which is expressed in oocytes from the primary stage of follicular development until ovulation (McGrath et al., 1995; Laitinen et al., 1998). GDF9B is closely related to GDF9 (Dube et al., 1998; Laitinen et al., 1998) and is expressed in mouse oocytes at the same time as GDF9, but in human primary follicles slightly later than GDF9. In the ovary GDF9 and GDF9B have now been shown to be expressed in the developing oocyte in humans (Aaltonen et al., 1999), rodents (Laitinen et al., 1998; Dube et al., 1998; Jaatinen et al., 1999), ruminants (Bodensteiner et al., 1999; Bodensteiner et al., 2000; Galloway et al., 2000) and marsupials (Eckery et al., 2002). In sheep expression of GDF9 can be seen in primordial follicles whereas GDF9B is expressed in primary follicles (Bodensteiner et al., 1999; Galloway et al., 2000).

GDF9 and GDF9B, like most other members of the TGFβ family, are coded as prepropeptides containing a signal peptide, a proregion and a C-terminal mature region which is the biologically active peptide. Cleavage of the mature region from the proregion is carried out by an intracellular furin-like protease, and occurs at a conserved furin protease cleavage site. Most members of the TGFβ superfamily are biologically active as dimers, and although GDF9 and GDF9B do not contain the cysteine molecule responsible for covalent interchain disulphide bonding seen in nearly all members of the family, these molecules are thought to be biologically active as dimers (Galloway et al., 2000; Yan et al., 2001). However it is unclear whether the physiologically active dimers are homodimers (GDF9-GDF9 and GDF9B-GDF9B), or heterodimers (GDF9-GDF9B) or whether all three dimer forms play a role. It has been postulated based on the above models that GDF9 homodimers play a more important role in the mouse but in sheep the GDF9B homodimers are the most bioactive (Yan et al., 2001). It is unclear whether any such difference is related to the fact that sheep are mono-ovulatory animals (maturing usually only one egg per cycle) whereas mice are poly-ovulatory. Clearly both GDF9 and GDF9B play crucial roles in controlling and maintaining fertility in mammals, and understanding the nature of their actions is essential for the development of therapies.

Jeffery et al., 2003, used a bioinformatics tool (GoCore) to analyse conserved regions across a number of different TGF-β family members and for GDF-9 and GDF-9B across different species, in an attempt to identify regions of functional significance. However, this study did not correlate regions identified as possible functionally significant sites with effects on modulation of ovulation in vivo.

It is an object of the present invention to identify domains of functional significance on the GDF-9 and/or GDF-9B molecules and to identify agonists and antagonists which interact with such domains or mimic such domains to modulate their biological activity thereby modulating mammalian ovarian function and ovulation rates in vivo; and/or to provide the public with a useful choice.

The term “comprising” as used in this specification and claims means “consisting at least in part of”, that is to say when interpreting independent claims including that term, the features prefaced by that term in each claim all need to be present but other features can also be present.

### SUMMARY OF THE INVENTION

The present invention is based on the identification of a novel domain of functional significance on the GDF-9 and GDF-9B molecules and to agonists and antagonists that interact therewith.

More specifically, the present invention is based on the identification and characterisation of a domain of functional significance around the N-terminal end of the mature GDF-9 and GDF-9B molecules, wherein the N-terminal domain of GDF-9 comprises the amino acid sequence:

| | |
|---|---|
| DQESASSELKKPLVPASVNLSEYFKQFLFPQNEC; | (SEQ ID NO: 1) |

and the N-terminal domain of GDF-9B comprises the amino acid sequence:

| | |
|---|---|
| QAGSIASEVPGPSREHDGPESNQC. | (SEQ ID NO: 2) |

The present invention is thus directed to an isolated fragment of the GDF-9 N-terminal domain, having the ability to modulate ovulation in a female mammal, wherein said GDF-9 N-terminal domain consists of the amino acid sequence DQESASSELKKPLVPASVNLSEYFKQFLFPQNEC (SEQ ID NO: 1), or a functional derivative, homolog, analog or mimetic thereof.

Preferably, the peptide fragment comprises at least one amino acid from positions 1-9 or 25-34 of SEQ ID NO: 1.

The present invention is also directed to an isolated peptide fragment of the GDF-9B N-terminal domain, having the ability to modulate ovulation in a female mammal, wherein said GDF-9B N-terminal domain consists of the amino acid sequence QAGSIASEVPGPSREHDGPESNQC (SEQ ID NO: 2), or a functional derivative, homolog, analog or mimetic thereof.

Preferably the peptide fragment comprises at least one amino acid from positions 1-5 or 21-23 of SEQ ID NO: 2.

The amino acid symbols correspond to the one letter code as set out in Table 1, below.

The isolated peptide fragments of the invention preferably comprise at least 5 contiguous amino acids of SEQ ID NO: 1 or SEQ ID NO: 2, or a functional derivative, homolog, analog or mimetic thereof. More preferably, the peptide fragments comprise at least 8, at least 10, at least 12, at least 14, at least 16, at least 18 or at least 20 contiguous amino acids of SEQ ID NO: 1 or SEQ ID NO: 2, or a functional derivative, homolog, analog or mimetic thereof.

Most preferably the peptide fragments of the invention are selected from the group comprising:

| | |
|---|---|
| DQESASSELKKPLV(C); | (SEQ ID NO: 3) |
| SEYFKQFLFPQNEC; | (SEQ ID NO: 4) |
| QAGSIASEVPGPSR(C); | (SEQ ID NO: 5) |
| and | |
| SREHDGPESNQC; | (SEQ ID NO: 6) |

or a functional derivative, homolog, analog or mimetic thereof.

Amino acids in parentheses refer to amino acids which are optionally added for conjugation purposes.

The present invention is also directed to an antibody or antibody fragment that binds to one or more of the peptide fragments of the invention.

In a further aspect the present invention provides a method of modulating the ovulation rate of a female mammal, said method comprising the step of administering to said mammal an effective amount of one or more isolated peptide fragments and/or antibodies or antibody fragments of the invention that are capable of interacting with the N-terminal domain of GDF-9 and/or GDF-9B as defined above, and altering the biological activity thereof.

Preferably, the invention provides a method of modulating the ovulation rate of a female mammal, comprising administering to said mammal an effective amount of one or more peptides selected from SEQ ID NOs 3-6, or a functional variant thereof, and/or an antibody or antibody fragment that binds thereto.

In a further aspect the present invention provides a use of one or more isolated peptide fragments of the invention and/or an antibody or antibody fragment that binds thereto, in the manufacture of a medicament for modulating the ovulation rate of a female mammal.

Preferably, the invention provides a use of one or more peptides selected from the group comprising SEQ ID NOs 3-6 or a functional variant thereof, and/or an antibody or antibody fragment that binds thereto, in the manufacture of a medicament of modulating the ovulation rate of a female mammal.

In a still further aspect, the present invention provides a pharmaceutical composition comprising one or more isolated peptide fragments of the invention that are capable of interacting with the N-terminal domain of GDF-9 and/or GDF-9B defined above, together with a pharmaceutically acceptable carrier or excipient.

Preferably, the composition comprises one or more peptides selected from the group comprising SEQ ID NOs 3-6 or a functional variant thereof, together with a pharmaceutically acceptable carrier or excipient. The composition may additionally or alternatively comprise an antibody or antibody fragment that binds to one or more of said peptides, together with a pharmaceutically acceptable carrier or excipient.

### DESCRIPTION OF THE DRAWINGS

The invention will now be described in more detail with reference to the figures of the accompanying drawings in which:

FIG. 1 shows the position of peptides of SEQ ID NOs 3 and 4 on GDF-9;

FIG. 2 shows the position of peptides of SEQ ID NOs 5 and 6 on GDF-9B;

FIGS. 3a and 3b show the sequence homology of GDF9 and GDF-9B from a number of different species and the N-terminal domain consensus sequences;

FIG. 4 shows the effect of treatment with GDF-9 and GDF-9B peptides on antral follicle development in bovine. (A=control, keyhole limpet hemocyanin (KLH) alone; B=KLH-GDF-9 (SEQ ID NO: 7); C=KLH-GDF-9B (SEQ ID NO: 5); D=KLH-GDF-9 and KLH-GDF-9B (SEQ ID NOS: 5 and 7);

FIG. 5 shows the effect of a polyclonal antibody from sheep immunized against ovine GDF-9 peptide SEQ ID NO 3 or SEQ ID NO 4, or GDF-9B peptide SEQ ID NO 5 on ovine (o) GDF-9 and oGDF-9B stimulated ³H-thymidine incorporation of rat granulosa cells when added directly to the bioassay. The horizontal line indicates the level of response of the granulosa cells treated with control media (i.e. media without oGDF-9 or oGDF-9B). Data presented are the mean and standard error of the mean of 4 replicate experiments. *P<0.05, **P<0.01 versus control media treated cells, ^{a}P<0.05, ^{b}P<0.01 versus oGDF-9+oGDF-9B treated cells that did not receive antibodies;

FIG. 6 shows the effect of a polyclonal antibody from sheep immunized against ovine GDF-9 peptide SEQ ID NO 4 on murine (m) GDF-9 and ovine (o) GDF-9B stimulated ³H-thymidine incorporation of rat granulosa cells when added directly to the bioassay. The horizontal line indicates the level of response of the granulosa cells treated with control media (i.e. media without mGDF-9 or oGDF-9B). Data presented are the mean and standard error of the mean of 3 replicate experiments. *P<0.05, **P<0.01 versus control media treated cells, ^{b}P<0.01 versus mGDF-9+oGDF-9B treated cells that did not receive antibodies;

FIG. 7 shows the effect of differing doses of a polyclonal antibody from sheep immunized against ovine GDF-9 peptide SEQ ID NO 4 on ovine (o) GDF-9 and oGDF-9B stimulated 3H-thymidine incorporation of rat granulosa cells when added directly to the bioassay. Data presented are the mean and standard error of the mean of 4 replicate wells from one pool of granulosa cells; and

FIG. 8 shows the effect of differing doses of a polyclonal antibody from sheep immunized against ovine GDF-9B peptide SEQ ID NO 5 on ovine (o) GDF-9 and oGDF-9B stimulated ³H-thymidine incorporation of rat granulosa cells when added directly to the bioassay. Data presented are the mean and standard error of the mean of 4 replicate wells from one pool of granulosa cells.

### DETAILED DESCRIPTION OF THE INVENTION

The present invention provides a novel domain of functional significance at the N-terminal end of GDF-9 and GDF-9B. It is postulated that stimulation or inhibition of this domain by agonists or antagonists that interact therewith will be effective in modulating the ovulation rate of a female mammal.

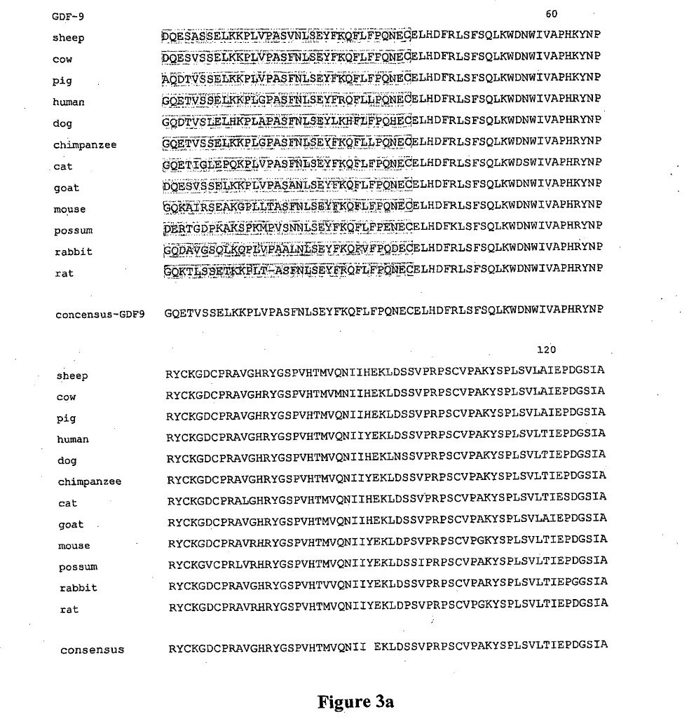

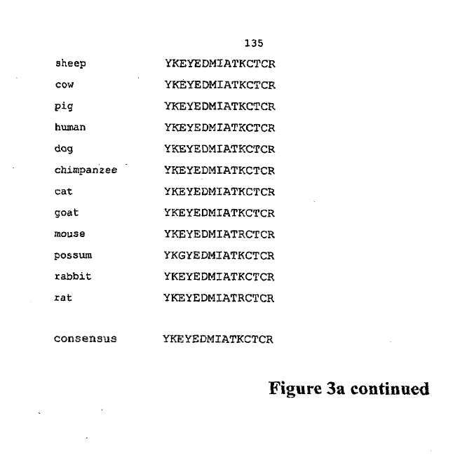

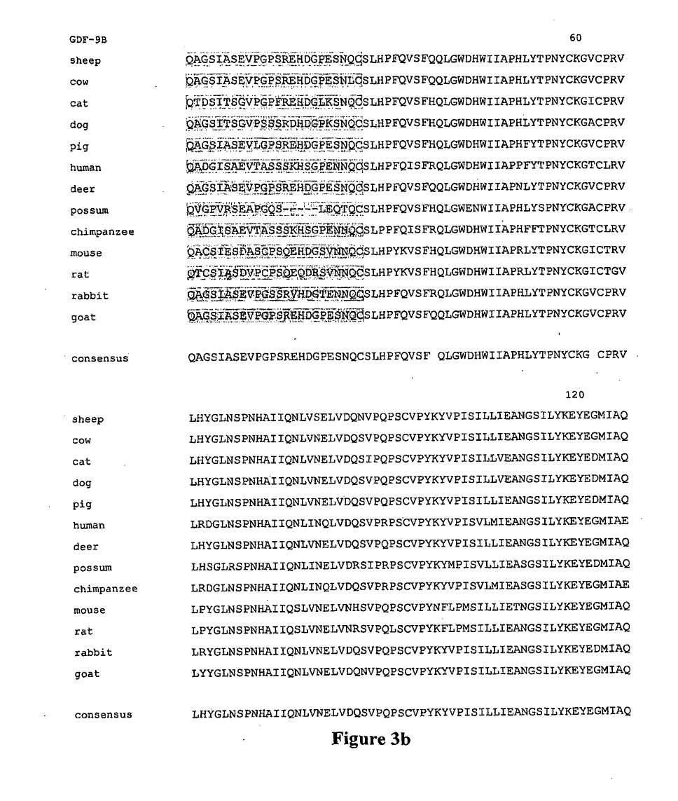

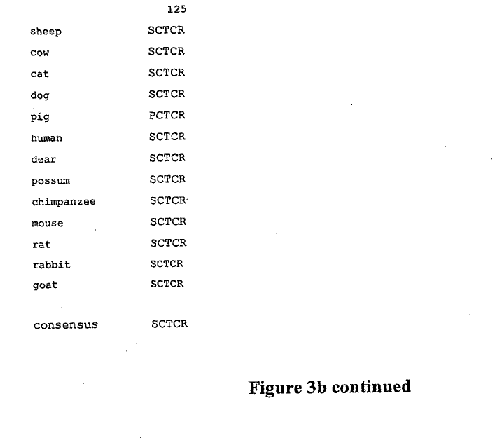

The sequences of the N-terminal domains were compared with the corresponding sequences of the GDF-9 and GDF-9B proteins in a number of different species and a consensus sequence determined as shown in FIGS. 3a and 3b for GDF-9 and GDF-9B respectively.

A number of peptides were then synthesised which corresponded with sequences within the N-terminal domains and which were anticipated to have an agonistic or antagonistic effect on the biological activity of GDF-9 and/or GDF-9B when administered in vivo. In particular, peptides were designed to be on the outside of the molecule according to its three dimensional structure; in a flexible region of the molecule; at least nine amino acids in length; non homologous with other TGF beta family members; non convergent with other known proteins; in areas that did not contain a glycosylation site; and so that they could be coupled to a carrier protein. It was considered that the combination of these factors would result in peptides that would be highly specific across species and would not have cross-reactivity problems.

Thus, in a first embodiment, the present invention is directed to an isolated fragment of the GDF-9 N-terminal domain, having the ability to modulate ovulation in a female mammal, wherein said GDF-9 N-terminal domain consists of the amino acid sequence DQESASSELKKPLVPASVNLSEYFKQFLFPQNEC (SEQ ID NO: 1). The amino acid symbols correspond to the one letter code as set out in Table 1, below.

Preferably, the isolated peptide fragment comprises at least one amino acid for positions 1 to 9 or 25 to 34 of SEQ ID NO: 1.

Preferably the peptide comprises at least 5 contiguous amino acids of SEQ ID NO: 1, or a functional derivative, homolog, analog or mimetic thereof. More preferably, the peptide comprises at least 8, at least 10, at least 12, at least 14, at least 16, at least 18 or at least 20 contiguous amino acids of SEQ ID NO: 1, or a functional derivative, homolog, analog or mimetic thereof.

Most preferably the peptides is selected from the group comprising:

| | |
|---|---|
| DQESASSELKKPLV(C); | (SEQ ID NO: 3) |
| and | |
| SEYFKQFLFPQNEC; | (SEQ ID NO: 4) |

or a functional derivative, homolog, analog or mimetic thereof. Amino acids in parentheses refer to amino acids optionally added for conjugation purposes.

The invention is also directed to an antibody or antibody fragment that binds to one or more GDF-9 peptides of the invention.

In a second embodiment the present invention is directed to an isolated peptide fragment of the GDF-9B N-terminal domain, having the ability to modulate ovulation in a female mammal, wherein said GDF-9B N-terminal domain consists of the amino acid sequence:

| | |
|---|---|
| QAGSIASEVPGPSREHDGPESNQC, | (SEQ ID NO: 2) |

or a functional derivative, homolog, analog or mimetic thereof. The amino acid symbols correspond to the one letter code as set out in Table 1 below.

Preferably, the isolated peptide fragment comprises at least one amino acid from positions 1 to 5 or 21 to 23 of SEQ ID NO: 2.

Preferably, the peptide comprises at least 5 contiguous amino acids of SEQ ID NO: 2 or a functional derivative, analog, homolog or mimetic thereof. More preferably, the peptide comprises at least 8, at least 10, at least 12, at least 14, at least 16, at least 18 or at least 20 contiguous amino acids of SEQ ID NO: 2, or a functional derivative, homolog, analog or mimetic thereof.

Most preferably, the peptide is selected from the group comprising:

| | |
|---|---|
| QAGSIASEVPGPSR(C); | (SEQ ID NO: 5) |
| and | |
| SREHDGPESNQC; | (SEQ ID NO: 6) |

or a functional derivative, homolog, analog or mimetic thereof.

The invention is also directed to an antibody or antibody fragment that binds to one or more GDF-9B peptides of the invention.

Amino acids in parentheses refer to amino acids optionally added for conjugation purposes.

The peptides of the present invention may be synthesised using known technology.

Analogs, derivatives or variants of the peptides of the invention may include sequence modifications or non-sequence modifications. Non-sequence modifications can include acetylation, methylation, phosphomethylation, carboxylation or glycosylation.

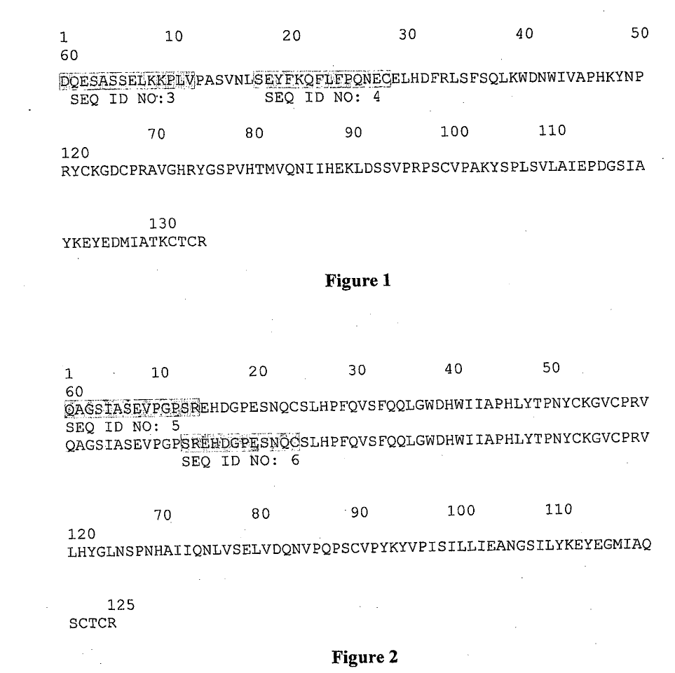

The specific N-terminal peptides exemplified in the present invention are shown in relation to their position on the N-terminal portion of GDF-9 and GDF-9B in FIGS. 1 and 2, respectively.

Preferred analogs include peptides who's sequence differs from those of the invention by one or more conservative amino acid substitutions, deletions or insertions which do not affect the biological activity of the peptide. Conservative substitutions typically include the substitution of one amino acid for another with similar characteristics, e.g., substitutions within the following groups: valine, glycine; glycine, alanine; valine, isoleucine, leucine; aspartic acid, glutamic acid; asparagine, glutamine; serine, threonine; lysine, arginine; and phenylalanine, tyrosine. Examples of conservative substitutions can also be found in the sequences of GDF-9 and GDF-9B in FIGS. 3a and 3b whereby the substitutions in different mammalian species compared to the consensus sequence are shown. Other conservative substitutions can be taken from Table 1 below.

TABLE 1
CONSERVATIVE AMINO ACID REPLACEMENTS

| For Amino | | |
|---|---|---|
| Acid | Code | Replace with any of |
| Alanine | A | D-Ala, Gly, beta-Ala, L-Cys, |
| | | D-Cys |
| Arginine | R | D-Arg, Lys, D-Lys, homo-Arg, |
| | | D-homo-Arg, Met, Ile, D-Met, |
| | | D-Ile, Orn, D-Orn |
| Asparagine | N | D-Asn, Asp, D-Asp, Glu, D-Glu, |
| | | Gln, D-Gln |
| Aspartic Acid | D | D-Asp, D-Asn, Asn, Glu, D-Glu, |
| | | Gln, D-Gln |
| Cysteine | C | D-Cys, S-Me-Cys, Met, D-Met, |
| | | Thr, D-Thr |
| Glutamine | Q | D-Gln, Asn, D-Asn, Glu, D-Glu, |
| | | Asp, D-Asp |
| Glutamic Acid | E | D-Glu, D-Asp, Asp, Asn, D-Asn, |
| | | Gln, D-Gln |
| Glycine | G | Ala, D-Ala, Pro, D-Pro, .beta.- |
| | | Ala Acp |
| Histidine | H | Asp, D-Asp, Lys, D-Lys, Tyr |
| Isoleucine | I | D-Ile, Val, D-Val, Leu, D-Leu, |
| | | Met, D-Met |
| Leucine | L | D-Leu, Val, D-Val, Leu, D-Leu, |
| | | Met, D-Met |
| Lysine | K | D-Lys, Arg, D-Arg, homo-Arg, |
| | | D-homo-Arg, Met, D-Met, Ile, |
| | | D-Ile, Orn, D-Orn |
| Methionine | M | D-Met, S-Me-Cys, Ile, D-Ile, |
| | | Leu, D-Leu, Val, D-Val |
| Phenylalanine | F | D-Phe, Tyr, D-Thr, L-Dopa, His, |
| | | D-His, Trp, D-Trp, Trans-3,4, or |
| | | 5-phenylproline, cis-3,4, or |
| | | 5-phenylproline |
| Proline | P | D-Pro, L-I-thioazolidine-4- |
| | | carboxylic acid, D- or L-1- |
| | | oxazolidine-4-carboxylic acid |
| Serine | S | D-Ser, Thr, D-Thr, allo-Thr, |
| | | Met, D-Met, Met(O), D-Met(O), |
| | | L-Cys, D-Cys |
| Threonine | T | D-Thr, Ser, D-Ser, allo-Thr, |
| | | Met, D-Met, Met(O), D-Met(O), |
| | | Val, D-Val |
| Tyrosine | Y | D-Tyr, Phe, D-Phe, L-Dopa, His, |
| | | D-His |
| Valine | V | D-Val, Leu, D-Leu, Ile, D-Ile, |
| | | Met, D-Met |

Other analogs include peptides with modifications which influence peptide stability. Such analogs may contain, for example, one or more non-peptide bonds (which replace the peptide bonds) in the peptide sequence. Also included are analogs that include residues other than naturally occurring L-amino acids, e.g. D-amino acids or non-naturally occurring synthetic amino acids, e.g. beta or gamma amino acids and cyclic analogs.

In a further aspect, the invention provides a use of the agonists and antagonists of the invention, in a method of modulating the ovulation rate of a female mammal, including human and non-human mammals. Such non-human mammals include sheep, cattle, goats, deer, pigs, horses, camelids, possums, non-human primates such as marmosets, cats, dogs and other commercially important species.

The method may comprise administering to said mammal an effective amount of one or more of said agonists or antagonists or a functional variant thereof, or an antibody or antibody fragment that binds thereto.

It is contemplated that equivalent N-terminal domain peptides of GDF-9 and GDF-9B of a particular mammal to be treated will be used in the methods of the present invention. Examples of GDF-9 and GDF-9B peptides from different species, and which correspond to the specific N-terminal peptides of SEQ ID NOs 3-6, are as shown in Table 1a, below.

TABLE 1A

| | Seq ID NO 5 | Seq ID No 6 |
|---|---|---|
| GDF-9B | QAGSIASEVPGPSR(C) | SREHDGPESNQC |
| cow | QAGSIASEVPGPSR | SREHDGPESNLC |
| deer | QAGSIASEVPGPSR | SREHDGPESNQC |
| dog | QAGSITSGVPSSSR | SRDHDGPKSNQC |
| cat | QTDSITSGVPGPFR | FREHDGLKSNQC |
| possum | QVGPVRSEAPGQS | S-----LEQTQC |
| chimpanzee | QADGISAEVTASSS | SSKHSGPENNQC |
| human | QADGISAEVTASSS | SSKHSGPENNQC |
| mouse | QACSIESDASCPSQ | SQEHDGSVNNQC |
| rat | QTCSIASDVPCPSQ | SQEQDRSVNNQC |
| rabbit | QAGSIASEVPGSSR | SRVHDGTENNQC |
| pig | QAGSIASEVLGPSR | SREHDGPESNQC |
| goat | QAGSIASEVPGPSR | SREHDGPESNQC |
| sheep | QAGSIASEVPGPSR | SREHDGPESNQC |
| Consensus | QAGSIASEVPGPSR | SREHDGPESNQC |
| | Seq ID NO 3 | Seq ID No 4 |
| GDF-9 | DQESASSELKKPLV(C) | SEYFKQFLFPQNEC |
| cow | DQESVSSELKKPLV | SEYFKQFLFPQNEC |
| dog | GQDTVSLELHKPLA | SEYLKHFLFPQHEC |
| chimpanzee | GQETVSSELKKPLG | SEYFKQFLLPQNEC |
| cat | GQETIGLEPQKPLV | SEYFKQFLFPQNEC |
| goat | DQESVSSELKKPLV | SEYFKQFLFPQNEC |
| human | GQETVSSELKKPLG | SEYFRQFLLPQNEC |
| mouse | GQKAIRSEAKGPLL | SEYFKQFLFPQNEC |
| sheep | DQESASSELKKPLV | SEYFKQFLFPQNEC |
| pig | AQDTVSSELKKPLV | SEYFKQFLFPQNEC |
| possum | DERTGDPKAKSPKM | SEYFKQFLFPENEC |
| rabbit | GQDAVGSQLKQPLV | SEYFKQFVFPQDEC |
| rat | GQKTLSSETKKPLT | SEYFRQFLFPQNEC |
| Consensus | GQETVSSELKKPLV | SEYFKQFLFPQNEC |

The amino acid alignment of mature GDF-9 and GDF-9B of a number of species is shown in FIGS. 3a and 3b. These alignment sequences were generated with the multiple alignment programme of Vector NTI. This programme uses the Clustal W algorithm (Thompson et al., 1994). The region encoding the mature region of the protein was identified and the nucleotide sequence was used to generate the predicted protein sequence. The sequences (GenBank accession number) used for alignment were as follows. GDF-9: cow (NM_{—}174681), goat (AH014112), sheep (AF078545), pig (NM_{—}001001909), dog (XM_{—}538624), cat (in house data base), possum (AY033826), rabbit (in house data base), mouse (NM_{—}008110), rat (NM_{—}021672), chimpanzee (XM_{—}527008) and human (NM_{—}005260). GDF-9B: cow (AY572412), deer (in house data base), dog (XM_{—}549005), cat (in house data base), possum (AH012378), chimpanzee (XM_{—}529247), human (AF082350), mouse (NM_{—}021670), rabbit (in house data base), pig (AF458070), goat (in house data base) and sheep (AF236079).

The modulation of the ovulation rate may comprise an increase or decrease in the ovulation rate of the female mammal by the administration of an agonist or antagonist of the invention to said animal resulting in antibodies being raised in vivo to said agonist or antagonist which in turn bind to the N-terminal domain of GDF-9 and/or GDF-9B to affect the biological activity thereof. Without being bound by theory, it is considered that binding of the N-terminal domain of GDF-9 and/or GDF-9B by an agonist/antagonist, and in particular by one or more antibodies raised against one or more peptides selected from the group comprising SEQ ID NOs: 3-6, results in altered circulating concentration of biologically active GDF-9 and/or GDF-9B. Where such a decrease comprises an approximate 50% fall in GDF-9B activity, an increase in ovulation rate has been observed previously, in particular, in Hanna Sheep where a single point mutation in the GDF-9B gene should result in half the amount of active GDF-9B in heterozygous animals and resulting in an increased ovulation rate and twinning. In homozygous animals, where there is no or very little circulating active GDF-9B, the animals were sterile (Galloway et al; 2000). Similarly, point mutations in the GDF-9 gene and concomitant modulation in ovulation rates have been observed in sheep (WO 03/102199; Hanrahan et al., 2003).

Thus, it is postulated that an agonist or antagonist of the invention that results in a decrease of approximately 50% in the circulating levels of active GDF-9 and/or GDF-9B will result in an increase in ovulation rate, whilst an agonist or antagonist that results in a reduction in the circulation concentration of active GDF-9 and/or GDF-9B to approximately zero, will result in a decrease in ovulation and sterilisation in a female mammal.

Preferably the agonist or antagonist of the invention is an antibody which binds to the consensus N-terminal domains of GDF-9 and/or GDF-9B of SEQ ID NOs: 1 and 2. It should be appreciated that the term “antibody” encompasses fragments or analogues of antibodies which retain the ability to bind to a consensus binding domain defined herein, including but not limited to Fv, Fc, F(ab)₂ fragments, ScFv molecules and the like. The antibody may be polyclonal or monoclonal, but is preferably monoclonal. Such antibodies may be prepared by any technique known in the art for example (Juengel et al., 2002) for administration to an animal, i.e. for use in passive immunisation. Alternatively, such antibodies may be produced in vivo by administration of an antigen in a suitable adjuvant, i.e. for use in active immunisation. Suitable adjuvants include Freund's complete or incomplete adjuvant, or similar immunostimulatory agent. The antibodies of the present invention may also be produced by genetic engineering methods such as chimeric and humanised monoclonal antibodies, comprising both human and non-human portions, which can be made using standard recombinant DNA techniques.

The present invention further contemplates the use of one or more agonists and/or antagonists of the present invention in combination with one or more active ingredients known to modulate ovulation rate to enhance the effect on ovulation. The active ingredients may be selected from the group comprising GDF-9; GDF-9B; BMPRII; BMPIB receptor (ALK6); ALK5 and BMP6; or functional fragments or variants thereof. In particular, the invention contemplates the use of BMP1B receptor in combination with an antibody or antibody fragment (Fc) that binds to a peptide of SEQ ID NOs: 5 or 6, in the modulation of ovulation in a female mammal.

The present invention further provides a pharmaceutical composition comprising at least one agonist or antagonist of the present invention together with a pharmaceutically acceptable carrier useful for the modulation of ovulation rate.

It is contemplated that the agonists or antagonists of the invention will be tested for biological activity in an animal model or in an in vitro model and suitably active compounds formulated into pharmaceutical compositions. The pharmaceutical compositions of the present invention may comprise, in addition to one or more agonists or antagonists of the present invention, a pharmaceutically acceptable excipient, carrier, buffer, stabiliser or other material well known in the art. Such materials should be non-toxic and should not interfere with the efficacy of the active ingredient. The precise nature of the carrier or other material will be dependent upon the desired nature of the pharmaceutical composition, and the route of administration e.g. oral, intravenous, cutaneous, subcutaneous, intradermal, intramuscular or intraperitoneal.

Pharmaceutical compositions for oral administration may be in tablet, lozenge, capsule, powder, granule or liquid form. A tablet or other solid oral dosage form will usually include a solid carrier such as gelatine, starch, mannitol, crystalline cellulose, or other inert materials generally used in pharmaceutical manufacture. Similarly, liquid pharmaceutical compositions such as a syrup or emulsion, will generally include a liquid carrier such as water, petroleum, animal or vegetable oils, mineral oil or synthetic oil.

For intravenous, cutaneous, subcutaneous, intradermal or intraperitoneal injection, the active ingredient will be in the form of a parenterally acceptable aqueous solution which is pyrogen-free and has suitable pH, isotonicity and stability.

In a further embodiment, the invention contemplates the use of one or more additional modulators of ovulation to be co-administered with the pharmaceutical composition of the present invention to give an additive or synergistic effect to the treatment regime. Examples of such additional modulators of ovulation include follicle stimulating hormone, Androvax (an androsteindione protein vaccine conjugate), and steroid hormone. Such modulators may be administered either separately, sequentially or simultaneously with at least one agonist or antagonist of the present invention depending upon whether ovulation is to be increased or decreased as will be appreciated by a skilled worker.

Administration of the pharmaceutical composition of the invention is preferably in a “therapeutically effective amount”, this being sufficient to show the desired benefit to the individual. The actual amount administered, and rate and time-course of administration, will depend on the nature and severity of the female mammals underlying condition. Prescription of treatment, e.g. decisions on dosage etc., is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 16th edition, Oslo, A. (ed), 1980.

The invention will now be described in greater detail by reference to specific Examples, which should not be construed as in any way limiting the scope of the invention.

### EXAMPLES

### Example 1

Use of peptides and antibodies to the N-terminal domains of GDF-9 and GDF-9B to manipulate ovulation in ovine.

Four 12 to 15 mer peptides were synthesised corresponding to the N-terminal domains of the GDF-9 and GDF-9B protein sequences and including, where necessary, additional residues to facilitate conjugation to Keyhole Limpet Haemocyanin (KLH) to generate an antigen. The peptide sequences were:

| | |
|---|---|
| DQESASSELKKPLV(C); | (SEQ ID NO: 3) |
| SEYFKQFLFPQNEC; | (SEQ ID NO: 4) |
| QAGSIASEVPGPSR(C); | (SEQ ID NO: 5) |
| and | |
| SREHDGPESNQC; | (SEQ ID NO: 6) |

In this study, groups of 10 anoestrous Romney ewes were injected with 0.4 mg/ewe of each peptide-KLH conjugate antigen in Freunds complete adjuvant and 10 anoestrous Romney ewes were injected with 0.4 mg/ewe KLH antigen as a control group. Subsequently at monthly intervals on 4 occasions, the animals were boosted with further antigen (0.2 mg/ewe on each occasion) in a Span/Tween/Oil mixture (sc) and oestrous activity monitored using vasectomised rams during the breeding season. The ovulation rate was assessed by laparoscopy over 4 successive oestrous cycles and again at the termination of the experiment approximately 20-30 days later. The mean ovulation rate was determined by averaging these 5 observations for each ewe.

All of the control ewes displayed cyclical oestrous activity. The results of the effect of peptides of SEQ ID NOs: 3-6 on ovulation rate show a shutdown of ovulation rate in the peptide treated ewes compared to the control ewes which correlated with a significant increase in the levels of anti-GDF-9 and anti-GDF-9B serum antibodies as shown below in Table 2. Antibody levels were measured using an ELISA procedure after sheep plasma was diluted 1:20,000 as previously described (Juengel et al., 2002).

Peptides of SEQ ID NOs: 3-5 resulted in shutdown of ovulation rate in the majority of animals within 2 weeks after the first booster injection. The shutdown of ovulation rate was maintained until the termination of the experiment. The peptide of SEQ ID NO:6 did not achieve significant shutdown of ovulation rate until the third observation (after 3 booster injections) and only achieved shutdown in 50% of animals. However, by the last booster injection, nine out of ten animals showed a shutdown in ovulation rate (Table 3). Shutdown of ovulation was measured by observation of the ovary whereby the ovary lacked externally visible corpora lutea. Ewes that were deemed to be in oestrus as assessed by observation of the uterus were excluded from the results of Table 3.

TABLE 2

| | Ovulation Rate | Antibody Titre (mean ± SEM) | |
|---|---|---|---|
| Treatment | (mean ± SEM) | Pre-treatment | Post Treatment |
| KLH (control) | 1.26 ± 0.08 | 0.023 ± 0.004 (GDF9) | 0.058 ± 0.030 (GDF9) |
| | | 0.019 ± 0.003 (BMP15) | 0.040 ± 0.14 (BMP15) |
| KLH-Peptide (SEQ ID NO: 3) | 0.34 ± 0.23 ⁺⁺ | 0.024 ± 0.002 | 2.355 ± 0.337 *** |
| KLH-Peptide (SEQ ID NO: 4) | 0.16 ± 0.09 ⁺⁺⁺ | 0.030 ± 0.005 | 2.182 ± 0.250 *** |
| KLH-Peptide (SEQ ID NO: 5) | 0.08 ± 0.05 ⁺⁺⁺ | 0.035 ± 0.017 | 1.193 ± 0.272 ** |
| KLH-Peptide (SEQ ID NO: 6) | 1.32 ± 0.37 | 0.018 ± 0.002 | 2.621 ± 0.244 *** |

⁺⁺ P < 0.01,
⁺⁺⁺ P < 0.001 using Mann-Whitney Test when compared to KLH(control)
** P < 0.01,
*** P < 0.001 pre-treatment versus post treatment within a treatment using paired T-test

TABLE 3
Number of ewes that showed inactive ovary/number of ewes examined.

| | Observation | | | | |
|---|---|---|---|---|---|
| Treatment | 1 | 2 | 3 | 4 | 5 |
| KLH (Control) | 0/10 | 0/8 | 0/9 | 0/9 | 0/10 |
| KLH-Peptide (SEQ ID NO: 3) | 8/10*** | 8/10*** | 8/10*** | 10/10*** | 10/10*** |
| KLH-Peptide (SEQ ID NO: 4) | 8/10*** | 9/10*** | 10/10*** | 10/10*** | 10/10*** |
| KLH-Peptide (SEQ ID NO: 5) | 8/10 | 10/10*** | 10/10*** | 10/10*** | 10/10*** |
| KLH-Peptide (SEQ ID NO: 6) | 1/10 | 2/10 | 5/10* | 5/10* | 9/10*** |

*P < 0.05,
***P < 0.001 using χ² when compared to KLH (Control)

These data show unequivocally that a significant decrease in ovulation rate can be induced by the administration of a peptide antigen targeted to the N-terminal domain of GDF-9 or GDF-9B. In this particular example, the effect was caused by active immunisation (i.e. administration of an antigen to raise antibodies in vivo). However, it is predicted that a similar effect will be seen by passive immunisation, i.e. by administration of the antibodies per se.

### Example 2

Use of peptides and antibodies to the N-terminal domains of GDF-9 and GDF-9B to manipulate ovulation in bovine.

Two 15-mer peptides were synthesised corresponding to the N-terminal domains of the bovine GDF-9 and GDF-9B protein sequence and including, wherein necessary, additional residues to facilitate conjugation to KLH to generate an antigen. The peptide sequences were:

| | |
|---|---|
| DQESVSSELKKPLV(C); | (SEQ ID NO: 7) |
| and | |
| QAGSIASEVPGPSR(C). | (SEQ ID NO: 5) |

Note that SEQ ID NO: 7 is a functional variant of SEQ ID NO: 3, comprising a single amino acid change (A→V at amino acid position 5).

In this study, groups of 10 Friesian-cross heifers were injected with 0.4 mg/heifer of each peptide-KLH conjugate antigen in 2 ml Freund's complete adjuvant given as four 0.5 ml injections; 10 heifers were injected with 0.4 mg/heifer of both peptide-KLH conjugate antigens and 10 heifers were injected with KLH antigen as a control group. Subsequently at monthly intervals on 2 occasions, the animals were boosted with further antigen (0.2 mg/heifer on each occasion) in a Span/Tween/Marcol mixture (SC). Animals were slaughtered approximately two weeks after the second booster immunisation and the ovaries collected. Ovarian activity was determined by visual examination of the ovary. Ovulation rate was assigned based on the number of corpora lutea visible on the surface of the ovary. Corpora lutea were determined to be regressed (and thus not counted in ovulation rate score) if it appeared non functional by morphological criteria (small, lacking vascularization, pale colour) with an additional functional corpora lutea present or was non functional by morphological (small, lacking vascularization, pale colour) and endocrinological criteria (low levels of progesterone in blood samples collect at time of ovarian collection and 2 weeks previously). Follicular development was assessed on 5 μm histological sections stained with hematoxylin and eosin.

Peripheral venous blood samples were collected (10 ml by heparinised vaccutainer) at the times of primary and first and second booster and also at 12 days after the first booster and again just before slaughter. The plasma samples were analyzed for antibody titres and progesterone concentrations.

All heifers appeared to have normal ovarian activity prior to immunization. Treatment with peptide of SEQ ID NO: 5, either alone or together with peptide of SEQ ID NO: 7 caused a significant alteration of ovarian activity as measured by ovulation rate. Treatment with peptide of SEQ ID NO: 7 alone showed twin ovulations of 30%, which is abnormally high, although this was not statistically significant with the number of animals examined. It is interesting to note that treatment with peptide of SEQ ID NO: 5 alone resulted in three animals having ovarian shutdown, i.e. a significant decrease in ovulation rate, similar to the results seen in sheep (see Table 4, below).

TABLE 4

| | Ovulation rate | | | | |
|---|---|---|---|---|---|
| Treatment | 0 | 1 | 2 | ≧3 | χ² |
| KLH (control) | 0 | 9 | 1 | 0 | |
| KLH-Peptide (SEQ ID NO: 5) | 3 | 3 | 4 | 0 | <0.01 |
| KLH-Peptide (SEQ ID NO: 7) | 0 | 7 | 3 | 0 | NS |
| KLH-Peptide (SEQ ID NO: 5) | 0 | 4 | 2 | 4 | <0.05 |
| and KLH-Peptide (SEQ ID NO: 7) | | | | | |

For χ² analysis, animals were assigned as normal (ovulation rate of 1) or affected (ovulation rate of 0 or ≧2) with each treatment group compared to KLH-control group.
NS = not significantly different.

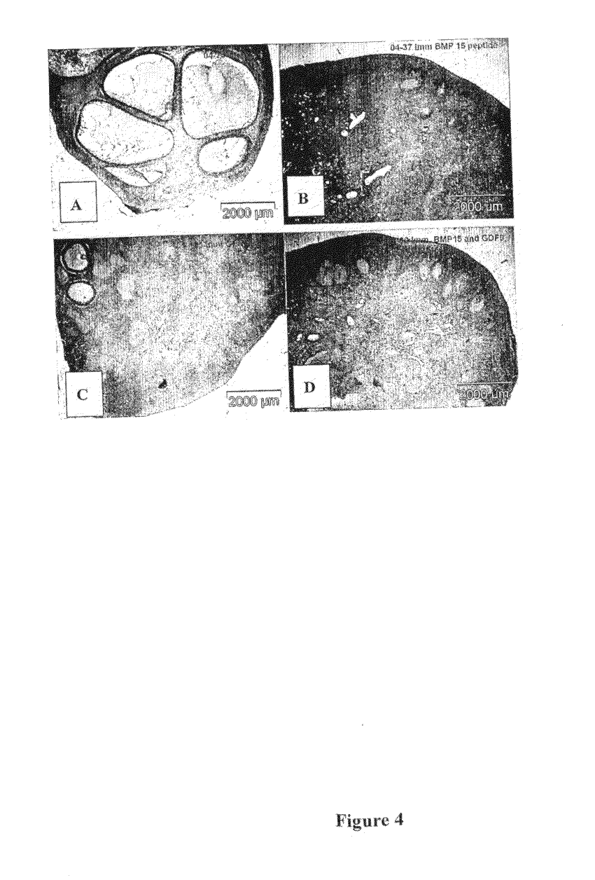

At collection, ovaries from heifers immunized with GDF-9B peptide (SEQ ID NO: 5) conjugated to KLH, GDF-9 peptide (SEQ ID NO 7) conjugated to KLH or the combination of GDF-9 peptide (SEQ ID NO: 7) conjugated to KLH and GDF-9B peptide (SEQ ID NO: 5) conjugated to KLH were noted to be morphologically different from the ovaries from the KLH immunized control heifers. The GDF-9 and/or GDF-9B immunized heifers had ovaries with fewer surface visible antral follicles. Similar observations were made when comparing histological sections of the ovaries (see FIG. 4). This observation was quantified on 5 μm histological sections stained with hemotoxylin and eosin whereby the percentage of the total area of the section occupied by antral space, the number of antral follicles per unit area and the average size (area) of the follicle was measured (see Table 5, below). Treatment with peptide of SEQ ID NO 5, SEQ ID NO 7 or both, decreased (P<0.01) the total area of the section occupied by antral space, the number of antral follicles per unit area and the average size of the follicle (see Table 5, below). It thus appears from these results that the ovulation rate would decrease if immunisation with the peptide fragments of the invention were to be continued for a longer term. Indeed, some animals treated with peptide of SEQ ID NO: 5 (GDF-9B), demonstrated an increased incidence of ovarian shutdown compared to control (see Table 4, above).

Effects of immunization against keyhole limpet haemocyanin (KLH), growth differentiation factor 9 (GDF9; SEQ ID NO 7: DQESVSSELKKPLV(C)) peptide conjugated to KLH, growth differentiation factor 9B (GDF-9B, SEQ ID NO 5; QAGSIASEVPGPSR(C)) conjugated to KLH and GDF-9 peptide conjugated to KLH+GDF-9B peptide conjugated to KLH on the percentage of the tissue section area occupied by antral space, the total number of follicles corrected for area and the average follicle area.

TABLE 5

| | | Total | | | |
|---|---|---|---|---|---|
| | | number | % tissue | total number | |
| | Number of | of | section area | of follicles | average |
| | Ovaries | sections | occupied by | corrected for | follicle area |
| Treatment | examined | examined | antral space | area | (mm²) |
| KLH | 10 | 72 | 27.4 ± 2.0 | 7.3 ± 0.6 | 4.0 ± 0.5 |
| KLH-peptide | 8 | 47 | 1.0 ± 0.3** | 2.2 ± 0.4** | 0.4 ± 0.1** |
| (SEQ ID NO: 5) | | | | | |
| KLH-peptide | 8 | 46 | 1.1 ± 0.4** | 2.3 ± 0.6** | 0.4 ± 0.1** |
| (SEQ ID NO: 7) | | | | | |
| KLH-peptide | 9 | 66 | 1.6 ± 1.0** | 1.6 ± 0.3** | 0.8 ± 0.4** |
| (SEQ ID NO: 5) | | | | | |
| KLH-peptide | | | | | |
| (SEQ ID NO: 7) | | | | | |

**P < 0.01
values presented are means ± standard error of the mean

These results demonstrate that the N-terminal peptides are active in modulating ovulation and follicular development in bovine. The dose given was the same as that for sheep, however in bovine the overall result was an increase in ovulation rather than a decrease. This is likely due to the “sheep dose” resulting in a weak immunisation resulting in reduced circulating level of active GDF-9 and/or GDF-9B, of approximately 50% which, as discussed above, results in an increase in ovulation rate.

This was confirmed by antibody titre data via ELISA as previously described (Juengel et al 2002) with minor modifications, see Table 6, below. The antigen coated onto the microtitre plates was 200 ng ovine GDF-9 mature protein or 100 ng ovine GDF-9B mature protein. Antibodies were detected using a 1:20,000 dilution of a rabbit anti-bovine IgG. Sera from heifers was diluted to 1:500. This is a 40 times less dilution than was required for sheep sera in experiment 1, above, suggesting that the dose of GDF-9 and/or GDF-9B peptides given to bovine was a ‘weak’ dose.

However, as mentioned above, it is likely that, given the decrease in follicular development, a decrease in ovulation rate may be induced in bovine by continuing the immunisation regime over a longer period.

Average optical density readings of bovine sera collected pre-immunization or post-immunization (just prior to ovarian collection) when measure by ELISA to determine immunization response.

TABLE 6

| | Coating Antigen | | | |
|---|---|---|---|---|
| | GDF-9 | | GDF-9B | |
| Immunization | Pre- | Post- | Pre- | Post- |
| Treatment | immunization | immunization | immunization | immunization |
| KLH | 0.40 + 0.09 | 0.32 + 0.05 | 0.05 + 0.01 | 0.23 + 0.18 |
| (control) | | | | |
| KLH-peptide | 0.46 + 0.17 | 1.90 + 0.26* | 0.25 + 0.13 | 0.42 + 0.20 |
| (SEQ ID NO: 7) | | | | |
| KLH-peptide | 0.37 + 0.05 | 0.35 + 0.04 | 0.12 + 0.01 | 1.61 + 0.27* |
| (SEQ ID NO: 5) | | | | |
| KLH-peptide | 0.39 + 0.07 | 1.15 + 0.16* | 0.13 + 0.01 | 1.12 + 0.28* |
| (SEQ ID NO: 7) | | | | |
| KLH-peptide | | | | |
| (SEQ ID NO: 5) | | | | |

*p < 0.01 comparing pre-immunization to post immunization within a treatment using a paired T-test.

It is expected that a larger dose in bovine, i.e. sufficient to reduce circulation levels of active GDF-9 and/or GDF-9B to approximately zero, or immunisation for a longer time period, will significantly reduce the ovulation rate in bovine.

These data show unequivocally that a significant modulation of ovulation rate and antral follicle development can be induced by the administration of a peptide antigen targeted at the N-terminal domain of GDF-9 and/or GDF-9B, indicating that the N-terminal mature regions of GDF-9 and GDF-9B are important for biological activity in cattle. In this particular example, the effect was caused by active immunisation (i.e. administration of an antigen to raise antibodies in vivo). However, it is predicted that a similar effect will be seen by passive immunisation i.e. by administration of antibodies per se.

### Example 3

Effect of antibodies raised against GDF-9 and GDF-9B peptide fragments on 3H-thymidine incorporation by rat granulosa cells in vitro.

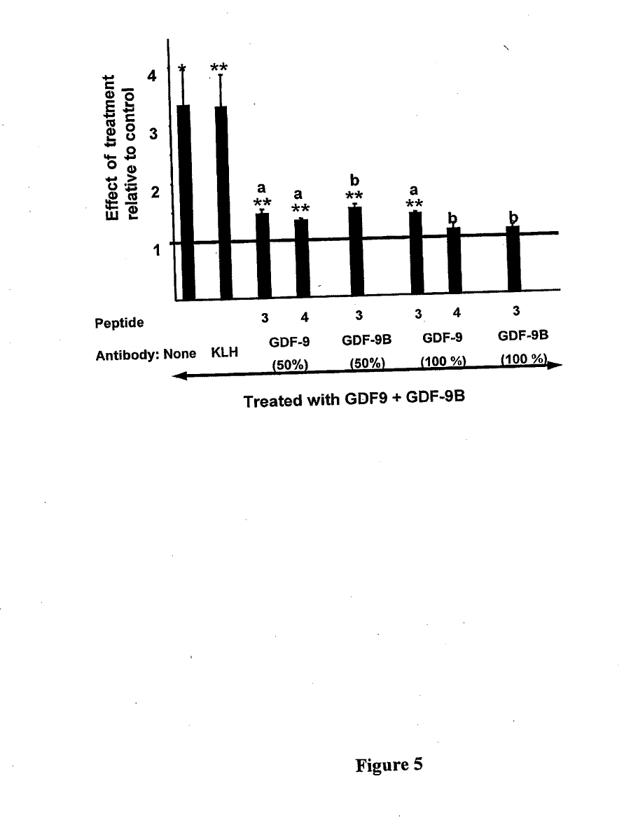

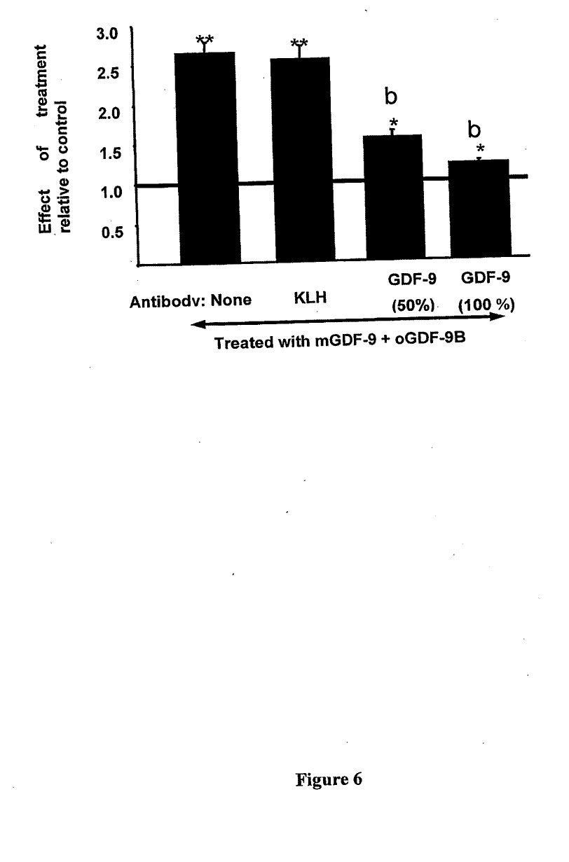

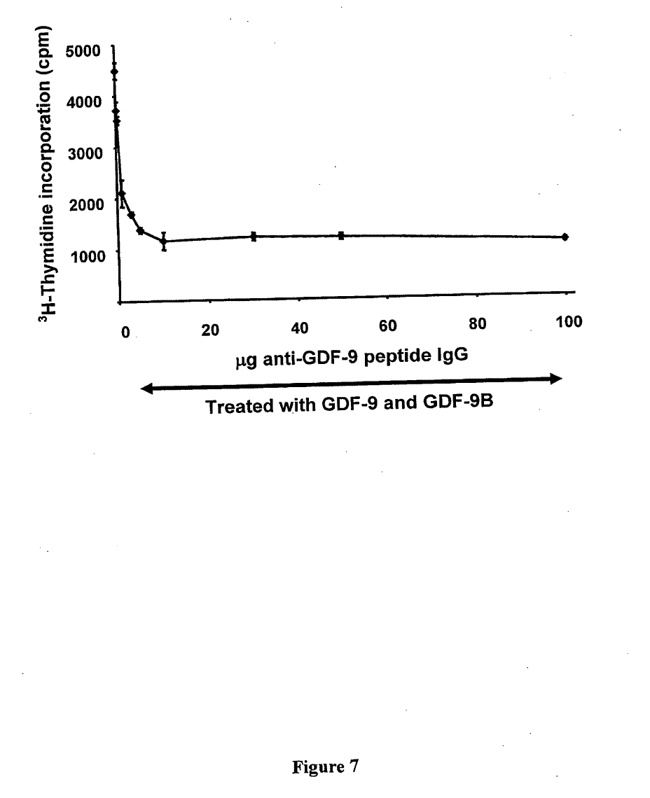

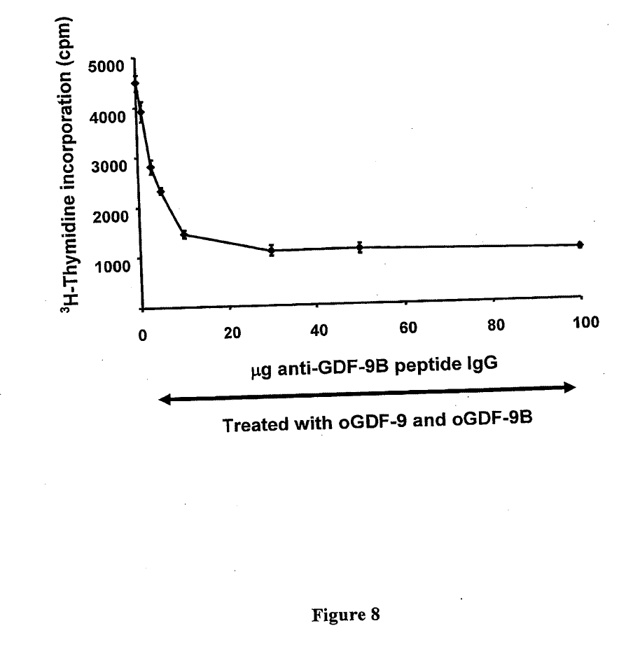

Various antibody preparations were tested for their ability to neutralize the effects of ovine or murine GDF-9 and ovine GDF-9B on rat granulosa cells when added directly to the granulosa cell culture using a previously described method (McNatty et al., 2005). A total of 100 μg/ml of IgG was added for each treatment which was comprised of IgG purified from sheep immunised with the GDF-9 or GDF-9B peptide fragments with the balance of IgG purified for sheep immunised with KLH. The antibodies were able to neutralize the effects of the growth factor that they were directed against (see FIGS. 5 and 6). Furthermore, this response was shown to be dose dependent in the two antibody samples tested against ovine GDF-9 and GDF-9B (P<0.001, see FIGS. 7 and 8).

Neutralization of the effects of ovine and murine GDF-9 and ovine GDF-9B by antibodies raised against peptide fragments of the N-terminal region of the protein (e.g. SEQ ID NO: 3, SEQ ID NO: 4, SEQ ID NO: 5) on incorporation of thymidine uptake by rat granulosa cells, taken together with the effects of active immunization with these peptides in sheep (see example 1) and cattle (see example 2), indicates that this region of the protein is important for biological activity in multiple species. These results also indicate that the antibodies against the N-terminal domain of the mature region of GDF-9 and GDF-9B are effective in passively neutralizing GDF-9 and GDF-9B.

It will be apparent to the person skilled in the art that while the invention has been described in some detail for the purposes of clarity and understanding, various modifications and alternatives to the embodiments and methods described herein may be made without departing from the scope of the invention as defined in the appended claims.

### INDUSTRIAL APPLICATION

The present invention provides compositions and methods for modulating the ovulation rate and therefore fertility in female mammals including humans.

### REFERENCES

1. McPherron A C, Lee S J: GDF-3 and GDF-9: two new members of the transforming growth factor-beta superfamily containing a novel pattern of cysteines. J Biol Chem 268:3444-3449, 1993.
2. McGrath S A, Esquela A F, Lee S J: Oocyte-specific expression of growth differentiation factor-9. Mol Endocrinol 9:131-136, 1995.
3. Laitinen M, Vuojolainen K, Jaatinen R, Ketola I, Aaltonen J, Lehtonen E, Heikinheimo M, Ritvos 0: A novel growth differentiation factor-9 (GDF-9) related factor is co-expressed with GDF-9 in mouse oocytes during folliculogenesis. Mech Dev 78:135-140, 1998.
4. Dube J L, Wang P, Elvin J, Lyons K M, Celeste A J, Matzuk M M: The bone morphogenetic protein 15 gene is X-linked and expressed in oocytes. Mol Endocrinol 12:1809-1817, 1998.
5. Jaatinen, R., Laitinen, M. P., Vuojolainen, K., Aaltonen, J., Louhio, H., Heikinheimo, K., Lehtonen, E. and Ritvos, 0: Localisation of growth differentiation factor-9 (Gdf-9) mRNA and protein in rat ovaries and cDNA cloning of rat GDF-9 and its novel homolog GDF-9B. Mol Cell Endocrinol 156: 189-193, 1999.
6. Aaltonen J, Laitinen M, Vuojolainen K, Jaatinen R, Horelli-Kuitunen N, Seppa L, Louhio H, Tuuri T, Sjoberg J, Butzow R, Hovatta O, Dale L, Ritvos O: Human growth differentiation factor-9 (GDF-9) and its novel homolog GDF-9B are expressed during early folliculogenesis. J Clin Endocrinol Metab 84: 2744-2750, 1999.
7. Bodensteiner, K. J., Clay, C. M., Moeller, C. L. and Sawyer, H. R.: Molecular cloning of the ovine growth/differentiation factor-9 gene and expression of growth/differentiation factor-9 in ovine and bovine ovaries Biology of Reproduction 60, 381-386, 1999.
8. Bodensteiner, K. J., McNatty, K. P., Clay, C. M., Moeller, C. L. and Sawyer, H. R.: Expression of growth and differentiation factor-9 in the ovaries of fetal sheep homozygous or heterozygous for the Inverdale prolificacy gene (FecX¹). Biology of Reproduction 62: 1479-1485, 2000.
9. Galloway S M, McNatty K P, Cambridge L M, Laitinen M P E, Juengel J L, Jokiranta T S, McLaren R J, Luiro K, Dodds K G, Montgomery G W, Beattie A E, Davis G H, Ritvos O: Mutations in an oocyte-derived growth factor gene (BMP15) cause increased ovulation rate and infertility in a dosage-sensitive manner. Nature Genetics 25:279-283, 2000.
10. Eckery, D. C., Whale, L. J., Lawrence, S. B., Wilde, K. A., McNatty, K. P. & Juengel, J. L.: Expression of mRNA encoding growth differentiation factor 9 and bone morphogenetic protein 15 during follicular formation and growth in a marsupial, the brushtail possum (Trichasurus vulpecula). Molecular & Cellular Endocrinology; 2002.
11. Yan, C., Wang, P., DeMayo, J., DeMayo, F. J., Elvin, J., Carino, C., Prasad, S. V., Skinner, S. S., Dunbar, B. S., Dube, J. L., Celeste, A. J. and Matzuk, M. M: Synergistic roles of bone morphogenetic protein 15 and growth differentiation factor 9 in ovarian function. Molecular Endocrinology 15: 854-866, 2001.
12. Fitzpatrick, S. L., Sindon, D. M., Shughue, P. J., Lane M. V., Merchenthaler, I. J., and Frail, D. F.: Expression of growth differentiating factor-9 messenger ribonucleic acid in ovarian and non-ovarian rodent and human tissue. Endocrinology, 139 (5), 2577-2578, 1998.
13. Hanrahan, J. P., Gregan, S. M., Mulsant, P., Mullen, M., Davis, G. M., Powell, R., Galloway, S. M.: Mutations in the genes for oocyte-derived growth factor GDF-9 and BMP-15 are associated with both increased ovulation rate and sterility in Cambridge and Belcare sheep (Ovis aeries). Biol. Reprod, 2003.
14. Juengel, J. L., Hudson, N. L., Heath, D. L., Smith, P., Reader, K. L., Lawrence, S. B., O'Connell, A. R., Laitinen, M. P., Cranfield, M., Groome, N. P., Ritvos, O., McNatty, K. P.: Growth differentiation factor 9 and bone morphogenic protein 15 are essential for ovarian follicular development in sheep. Biol. Reprod. 67(b), 1777-1789, 2002.
15. Jeffery, L., Mottershead, D. G., Myllymaa, S., Gilchrist, R., Groome, N., and Ritvos, O.: Grouping of conserved regions (GoCore): Better prediction of function from sequences. Poster ESHRE Campus April, 2003.
16. Thompson J D, Higgins D G, Gibson T J. CLUSTAL, W: Improving the sensitivity of progressive multiple sequence alignment through sequence weighting, position-specific gap penalties and weight matrix choice. Nucleic Acids Res. 1994 22:4673-4680.
17. McNatty, K. P., Juengel, J. L., Reader, K. L., Lun, S., Myllymaa, S., Lawrence, S. B., Western, A., Meerasahib, M. F., Mottershead, D. G., Groome, N. P., Ritvos, O., Laitinen, M. P. E. (2005). Bone morphogenetic protein 15 and growth differentiation factor 9 co-operate to regulate granulosa cell function. Reproduction 129:473-480.

All references are incorporated into this specification in their entirety by means of this reference.

1. An isolated peptide fragment of the GDF-9 N-terminal domain, having the ability to modulate ovulation in a female mammal, wherein said GDF-9 N-terminal domain consists of the amino acid sequence DQESASSELKKPLVPASVNLSEYFKQFLFPQNEC (SEQ ID NO: 1), wherein said fragment comprises at least 5 contiguous amino acids of SEQ ID NO: 1, or a functional derivative, homolog, analog or mimetic thereof.

2. An isolated peptide fragment as claimed in claim 1 comprising at least one amino acid from positions 1 to 9 or 25 to 34 of SEQ ID NO: 1.

3. An isolated peptide fragment of the GDF-9B N-terminal domain, having the ability to modulate ovulation in a female mammal, wherein said GDF-9B N-terminal domain consists of the amino acid sequence QAGSIASEVPGPSREHDGPESNQC (SEQ ID NO:
2), wherein said fragment comprises between 5 and 14 contiguous amino acids of SEQ ID NO: 2 and further comprises at least one amino acid from positions 1 to 6 or 22 to 24 of SEQ ID NO: 2, or a functional derivative, homolog, analog or mimetic thereof.

4. An isolated peptide fragment of the GDF-9 N-terminal domain, having the ability to modulate ovulation in a female mammal wherein said fragment comprises at least 5 contiguous amino acids of SEQ ID NO: 1 and further comprises at least one amino acid from positions 1 to 9 or 25 to 34 of SEQ ID NO: 1, or a functional derivative, homolog, analog or mimetic thereof.

5. An isolated peptide fragment of the GDF-9B N-terminal domain, having the ability to modulate ovulation in a female mammal, wherein said fragment comprises between 5 and 14 contiguous amino acids of SEQ ID NO: 2 and further comprises at least one amino acid from positions 1 to 6 or 22 to 24 of SEQ ID NO: 2 or a functional derivative, homolog, analog or mimetic thereof.

6. An isolated peptide fragment as claimed in claim 1, comprising at least 8 contiguous amino acids of SEQ ID NO: 1 or SEQ ID NO: 2, or a functional derivative, homolog, analog or mimetic thereof.

7. An isolated peptide fragment as claimed in claim 1, comprising at least 10 contiguous amino acids of SEQ ID NO: 1 or SEQ ID NO: 2, or a functional derivative, homolog, analog or mimetic thereof.

8. An isolated peptide fragment as claimed in claim 1, comprising at least 12 contiguous amino acids of SEQ ID NO: 1 or SEQ ID NO: 2, or a functional derivative, homolog, analog or mimetic thereof.

9. An isolated peptide fragment as claimed in claim 1, comprising at least 14 contiguous amino acids of SEQ ID NO: 1 or SEQ ID NO: 2, or a functional derivative, homolog, analog or mimetic thereof.

10. An isolated peptide fragment as claimed in claim 1, selected from the group comprising:

| | |
|---|---|
| DQESASSELKKPLV(C); | (SEQ ID NO: 3) |
| SEYFKQFLFPQNEC; | (SEQ ID NO: 4) |
| QAGSIASEVPGPSR(C); | (SEQ ID NO: 5) |
| and | |
| SREHDGPESNQC; | (SEQ ID NO: 6) |

or a functional derivative, homolog, analog or mimetic thereof.

11. An antibody or antibody fragment which binds to one or more peptide fragments of claim 1.

12. A method of modulating the ovulation rate in a female mammal, said method comprising the step of administering to said mammal an effective amount of one or more isolated peptide fragments as claimed in claim 1.

13. A method of modulating the ovulation rate in a female mammal, said method comprising the step of administering to said mammal an effective amount of one or more antibodies or antibody fragments of claim 11.

14. A method of modulating the ovulation rate in a female mammal, said method comprising the step of administering to said mammal an effective amount of one or more isolated peptide fragments as claimed in claim 1 together with one or more antibodies or antibody fragments which bind thereto.

15. A method of modulating the ovulation rate in a female mammal, comprising administering to said mammal an effective amount of one or more peptide fragments selected from SEQ ID NOs 3-6, or a functional derivative, homolog, analog or mimetic thereof, and/or an antibody or antibody fragment that binds thereto.

16. (canceled)

17. (canceled)

18. (canceled)

19. (canceled)

20. A pharmaceutical composition comprising one or more isolated peptide fragments as claimed in claim 1, together with a pharmaceutically acceptable carrier or excipient.

21. A pharmaceutical composition comprising one or more antibodies or antibody fragments as claimed in claim 11, together with a pharmaceutically acceptable carrier or excipient.

22. A pharmaceutical composition comprising one or more isolated peptide fragments as claimed in claim 1 and one or more antibodies or antibody fragments which bind thereto, together with a pharmaceutically acceptable carrier or excipient.

23. A pharmaceutical composition comprising one or more peptides selected from the group comprising SEQ ID NOs 3-6 or a functional variant thereof, and/or an antibody or antibody fragment that binds thereto, together with a pharmaceutically acceptable carrier or excipient.

24. A method of modulating the ovulation rate in a female mammal, comprising administering to said mammal an effective amount of a composition as claimed in any one of claims 20 to 23, in combination with one or more compounds selected from the group comprising GDF-9, GDF-9B, BMPRII, BMPIB receptor (ALK6), ALK5 and BMP6 or a functional fragment or variant thereof.

25. A method of modulating the ovulation rate in a female mammal comprising administering to said mammal an effective amount of an antibody or antibody fragment (Fc) that binds to a peptide of any one of SEQ ID NOS: 3 to 6, in combination with BMPIB receptor (ALK6).

26. A method of modulating the ovulation rate in a female mammal comprising administering to said mammal an effective amount of a composition as claimed in claim 20 in combination with a modulator of ovulation selected from the group consisting of FSH, androvax and steroid hormone.

27-29. (canceled)


SEQUENCE LISTING (7 sequences)

SEQ ID NO: 1 - protein, 34 aa, Ovis aries
DQESASSELKKPLVPASVNLSEYFKQFLFPQNEC

SEQ ID NO: 2 - protein, 24 aa, Ovis aries
QAGSIASEVPGPSREHDGPESNQC

SEQ ID NO: 3 - protein, 14 aa, Ovis aries
DQESASSELKKPLV

SEQ ID NO: 4 - protein, 14 aa, Ovis aries
SEYFKQFLFPQNEC

SEQ ID NO: 5 - protein, 14 aa, Ovis aries
QAGSIASEVPGPSR

SEQ ID NO: 6 - protein, 12 aa, Ovis aries
SREHDGPESNQC

SEQ ID NO: 7 - protein, 14 aa, Ovis aries
DQESVSSELKKPLV